(19) United States
(12) Patent Application Publication
(10) Pub. No.: US 2015/0209027 A1
(43) Pub. Date: Jul. 30, 2015

(54) Adjustable Suture Lock Loop

(71) Applicant: SUSPENSION ORTHOPAEDIC SOLUTIONS, LLC, Severna Park, MD (US)

(72) Inventors: Robert S. Collins, Severna Park, MD (US); Jeffrey Gelfand, Severna Park, MD (US); Steve Santangelo, Shelton, CT (US)

(73) Assignee: SUSPENSION ORTHOPAEDIC SOLUTIONS, LLC, Severna Park, MD (US)

(21) Appl. No.: 14/682,323

(22) Filed: Apr. 9, 2015

Related U.S. Application Data
(63) Continuation of application No. 13/470,745, filed on May 14, 2012, now abandoned.
(60) Provisional application No. 61/485,855, filed on May 13, 2011.

(51) Int. Cl. A61B 17/04

(52) U.S. Cl. CPC A61B 17/0401; A61B 2017/0496; A61B 2017/0414; A61B 2017/0459; A61B 2017/0464; A61B 2017/00004

(57) ABSTRACT

The present disclosure relates to an adjustable suture lock loop for use in soft tissue repair and, more particularly, an adjustable suture lock loop constructed from a suture material having a lumen and one or more anchors. The adjustable suture lock loop can include a strand of braided suture material having a first end and a second end, and a lumen; and one or more anchors, wherein each anchor has two or more holes disposed therethrough that are large enough to permit the suture to pass therethrough; and both ends of the suture material pass through the lumen of a first section of the suture material, and wherein the first end of the suture material is free, while the second end is secured to form a loop.

### CROSS-REFERENCE TO RELATED APPLICATION

This application claims priority to U.S. Provisional Application for Patent Ser. No. 61/485,855 filed May 13, 2011, the disclosure of which is incorporated herein by reference in its entirety.

### FIELD OF THE DISCLOSURE

The present disclosure relates to an adjustable suture lock loop for use in soft tissue repair and, more particularly, to an adjustable suture lock loop constructed from a suture material having a lumen and one or more anchors.

### BACKGROUND

Soft tissue repair procedures often involve the reattachment of soft tissue, such as tendons and ligaments, to bone. Currently available products for performing these procedures have various problems associated with their use. For example, there can be difficulties in determining the length of the suture running from the soft tissue to a securing device and in the process of tying knots in the suture. This can lead to over-tensioning of the suture or slack in the system resulting in unsatisfactory reattachment of the soft tissue. These problems and others are overcome by the devices described herein.

### SUMMARY OF THE DISCLOSURE

An adjustable suture lock loop according to the present disclosure can comprise a strand of braided suture material having a first end and a second end, and a lumen; and one or more anchors. Each anchor can have two or more holes disposed therethrough, and the holes are large enough to permit the suture to pass therethrough. In the suture lock loop, both ends of the suture material can pass through the lumen of a first section of the suture material, and the first end of the suture material can be free, and the second end can be secured to form a loop. The strand of suture material can have a second lumen through which the second end of the suture material is passed.

Application of longitudinal force to the suture that increases the length of the suture results in contraction of the lumen. Application of longitudinal force that decreases the length of the suture results in expansion of the lumen. Through application of longitudinal force, a surgeon can manipulate the adjustable suture lock loop to have a desired size and tension in order to properly secure soft tissue.

### BRIEF DESCRIPTION OF THE DRAWINGS

FIG. 1 shows an embodiment of the adjustable suture lock loop.

FIG. 2 shows one embodiment of an anchor for use in the adjustable suture lock loop of FIG. 1.

FIG. 3 shows an alternative embodiment of the adjustable suture lock loop.

### DETAILED DESCRIPTION

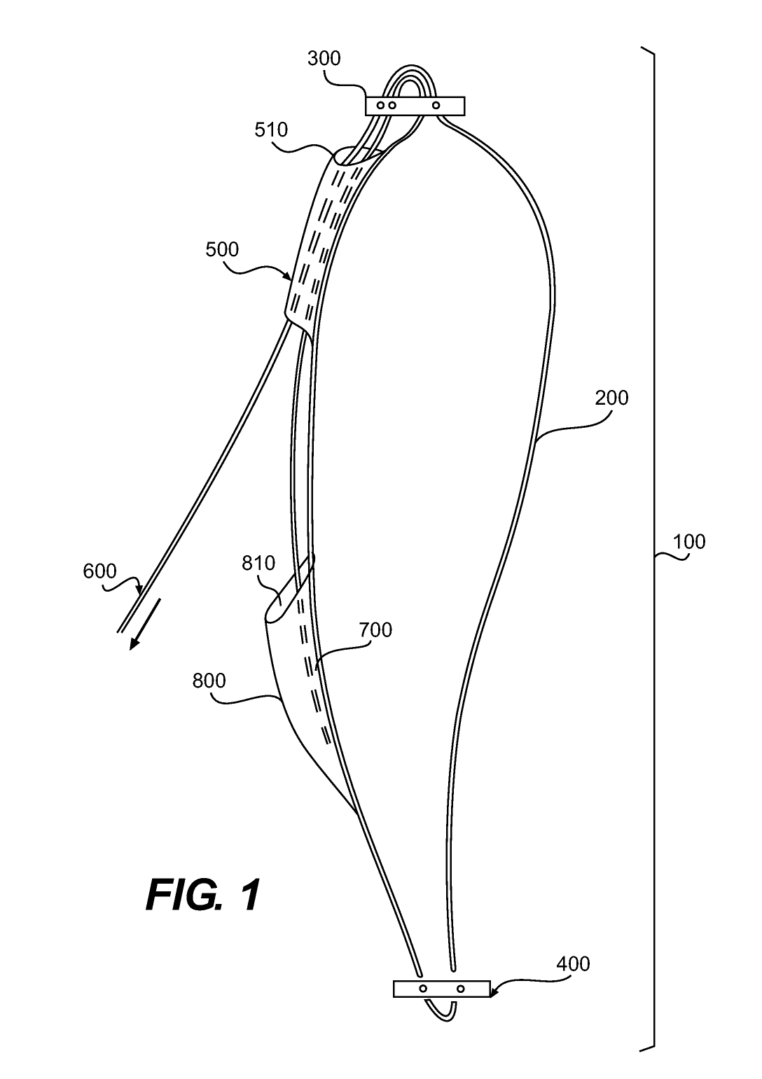

As shown in FIG. 1, an adjustable suture lock loop 100 may be constructed from a single strand of hollow braided suture material 200. The hollow braided suture may be constructed out of any suitable fiber, including natural fibers such as cotton or silk, or synthetic fibers such as polyester or polyethylene, or a blend of fibers, which blend may be a blend of natural fibers, a blend of synthetic fibers, or a mix of natural and synthetic fibers. The suture is braided, with an inner lumen. When longitudinal force is applied to the suture, increasing the length of the suture, the lumen contracts; when longitudinal force is applied that decreases the length of the suture, the lumen expands.

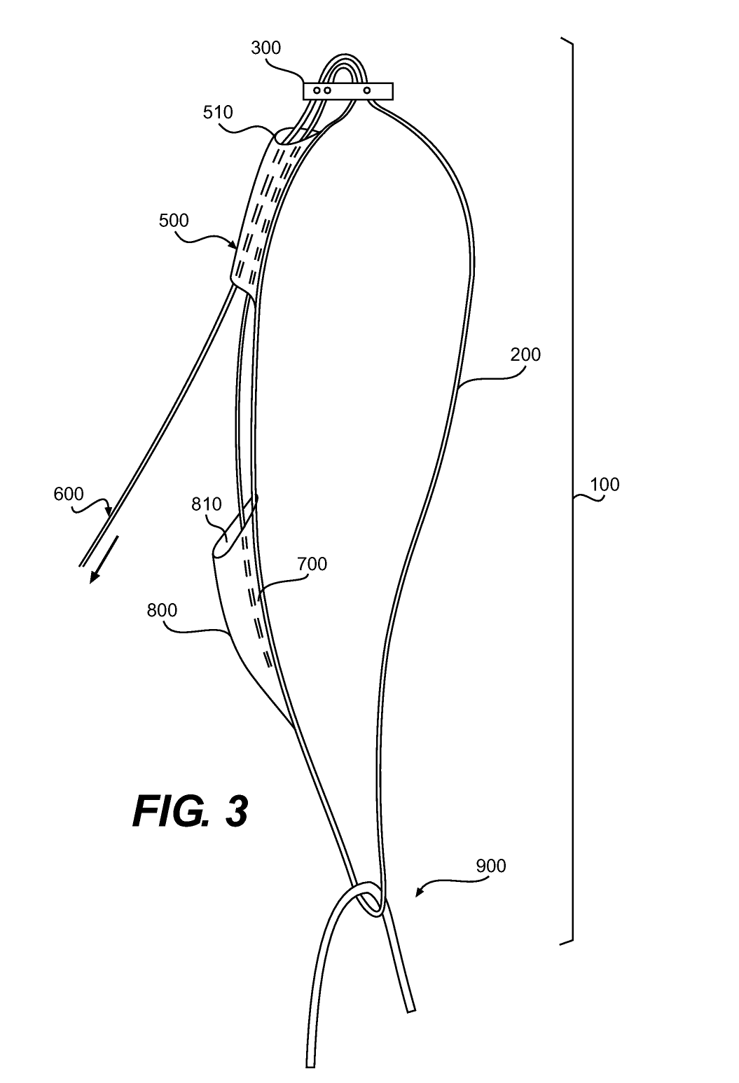

In the embodiment shown in FIG. 1, the strand of suture material 200 is engaged with a first anchor 300. FIG. 1 also shows the strand of suture material engaged with a second anchor 400; the use of two or more anchors is optional, at the discretion of the surgeon. FIG. 3 shows an embodiment in which there is only one anchor incorporated in the loop. A second portion of the loop may be used to secure a tendon or ligament (900), or a bone, graft or any other materials secured to such tissue.

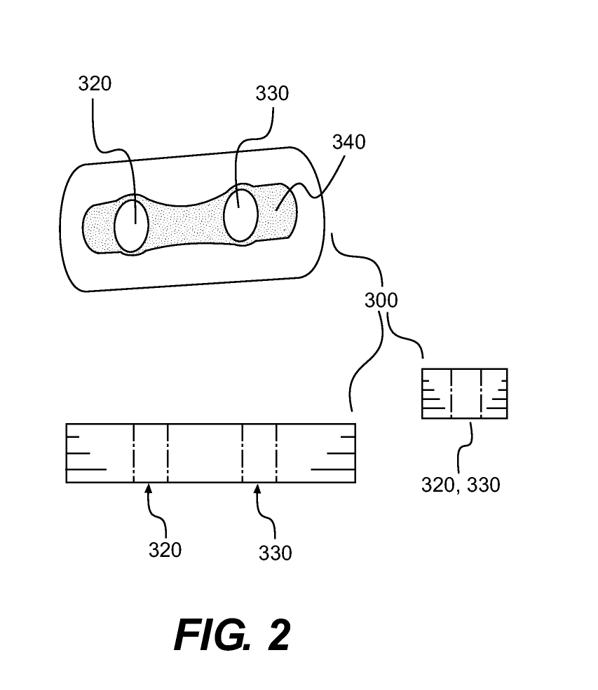

The anchors 300, 400 may comprise two or more holes through which the single strand of suture is threaded. An embodiment of an anchor which may be used in the adjustable suture lock loop is shown in FIG. 2. Although FIG. 2 refers to anchor 300, the anchors 300, 400 may be the same size, shape, and configuration, or may be different, as required. The anchor 300 of FIG. 2 comprises two holes 320, 330. The holes are of a size that is equal to or greater than the thickness of the suture material 200, such that the holes are large enough to permit the suture material 200 to pass through. The holes may be circular, elliptical, or of any other shape required.

The anchor also may comprise a channel 340 between and surrounding the holes 320, 330. The depth of the channel may be greater than, equal to, or less than the thickness of the suture material 200. A channel 340 may be disposed on one or both sides of the anchor 300. An anchor for use with the adjustable suture lock loop may be oval (as shown in FIG. 2), round, rectangular, or of any other suitable shape. The anchor may be dimensioned in width and thickness (i.e., the cross section) so that in one orientation it can pass through a hole drilled in a bone, for example, the radius bone of the arm, while in a deployed state/orientation, it resists passing back through the hole. In one embodiment, an oval anchor as shown in FIG. 2 may be about 10 mm long, about 4 mm wide, and about 1 mm thick. The size of the anchor may be modified as required. The anchor may be constructed of any suitable natural or synthetic biocompatible material, such as stainless steel or titanium, or a metal alloy, a ceramic such as zirconium oxide, or a plastic such as polyester. Where required, the anchor may be constructed of a bioabsorbable material.

In the embodiment shown in FIG. 1, the strand of suture material 200 is threaded through two anchors 300 and 400. The suture material 200 comprises a first end 600 and a second end 700. Both the first end 600 and the second end 700 pass through the lumen 510 of a first section 500 of the suture 200. The first end 600 remains free. In the embodiment shown in FIG. 1, the second end 700 is inserted into the lumen 810 of a second section 800 of the suture 200. When longitudinal force is applied to the second section 800, thereby increasing its length, the lumen 810 contracts, securing the second end 700 of the suture 200. In an alternative embodiment, the second end 700 is not inserted into the lumen 810, but instead is secured by an alternative means; for example, second end 700 may be woven back into the suture, or may be secured directly to the anchor.

The size of the loop may be decreased by pulling on the free end 600 of the suture 200, thereby bringing the anchors 300, 400 closer together. When the loop is at its desired size, further tension on the free end 600 of the suture 200 will cause the lumen 510 to contract, securing the suture within and stabilizing the size of the loop by preventing the suture from passing freely through the compressed lumen.

While the foregoing disclosure has been described in some detail for purposes of clarity and understanding, it will be appreciated by one skilled in the art from a reading of this disclosure that various changes in form and detail can be made without departing from the true scope of the disclosure and appended claims. All patents and publications cited herein are entirely incorporated herein by reference.

What is claimed is:

1. An adjustable suture lock loop comprising:
a strand of braided suture material comprising a first end and a second end, and a lumen; and
one or more anchors, wherein each anchor comprises two or more holes disposed therethrough, wherein the holes are large enough to permit the suture to pass therethrough;
wherein both ends of the suture material pass through the lumen of a first section of the suture material, and wherein the first end of the suture material is free, while the second end is secured to form a loop.

2. The suture lock loop of claim 1, wherein the suture material comprises a natural material.

3. The suture lock loop of claim 2, wherein the natural material is silk.

4. The suture lock loop of claim 1, wherein the suture material comprises a synthetic material.

5. The suture lock loop of claim 4, wherein the synthetic material is selected from the group consisting of polyester and polyethylene.

6. The suture lock loop of claim 1, wherein the suture material is bioabsorbable.

7. The suture lock loop of claim 1, wherein the anchor comprises a metal.

8. The suture lock loop of claim 7, wherein the metal is selected from the group consisting of stainless steel and titanium.

9. The suture lock loop of claim 1, wherein the anchor comprises a ceramic.

10. The suture lock loop of claim 9, wherein the ceramic is zirconium oxide.

11. The suture lock loop of claim 1, wherein the anchor comprises a synthetic material.

12. The suture lock loop of claim 11, wherein the synthetic material is selected from the group consisting of polyester and polyethylene.

13. The suture lock loop of claim 1, wherein the anchor is oval.

14. The suture lock loop of claim 1, wherein the holes are circular.

15. The suture lock loop of claim 1, wherein the anchor further comprises a channel between the holes.

16. The suture lock loop of claim 1, wherein the width and thickness of the anchor is small enough to pass through a hole drilled in a bone, and the length of the anchor is greater than the diameter of the hole, such that the anchor will not pass back through the bone when the loop is implanted in a patient.

17. The suture lock loop of claim 1, wherein the second end is disposed within the lumen of a second section of the suture material.

18. The suture lock loop of claim 1, wherein the second end is woven into the suture material.

19. The suture lock loop of claim 1, wherein the second end is secured directly to the anchor.
